(19) United States
(12) Patent Application Publication
(10) Pub. No.: US 2006/0184038 A1
(43) Pub. Date: Aug. 17, 2006

(54) System, method, and product for analyzing images comprising small feature sizes

(75) Inventors: David R. Smith, San Jose, CA (US); Albert K. Bukys, Lexington, MA (US); Simon Cawley, Oakland, CA (US); Daniel M. Bartell, San Carlos, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/289,975

(22) Filed: Nov. 30, 2005

Related U.S. Application Data
(60) Provisional application No. 60/631,645, filed on Nov. 30, 2004.

(51) Int. Cl. A61B 6/00

(52) U.S. Cl. 600/476

(57) ABSTRACT

A method of reconstructing a cell using a raw image of a biological probe array is described that comprises (a) assigning an intensity value to a reconstructed cell of a reconstructed image, where each reconstructed cell comprises a plurality of reconstructed pixels; (b) determining a weighted intensity value for each reconstructed pixel in the reconstructed cell using the intensity value of the reconstructed cell and a weight value; (c) determining an error value for each reconstructed pixel using the weighted intensity value and a raw intensity value corresponding to a pixel in the raw image; (d) updating the intensity value of the reconstructed cell using the error value; and (e) repeating steps (b)-(d) until convergence, wherein the intensity value for the reconstructed cell is representative of light emitted from a corresponding probe feature on the biological probe array.

### RELATED APPLICATIONS

The present application claims priority from U.S. Provisional Patent Application Ser. No. 60/631,645, titled “System, Method and Product for Analyzing Images Comprising Small Feature Sizes”, filed Nov. 30, 2004, which is hereby incorporated by reference herein in its entirety for all purposes.

### BACKGROUND

1. Field of the Invention

The present invention relates to systems and methods for examining biological material. In particular, the invention relates to the analysis of images from scanned biological probe arrays comprising probe features of very small size, such as for instance probe features that are 8 μm or less across. Accurate analysis of small features sizes becomes increasingly more complicated as the feature size becomes smaller where elements of the scanning system may contribute to sources of error in the resulting image. For example, the scanning system may implement a light source focused to a spot and scanned across the probe array where the size of the spot is large in comparison to the size of the probe features and inter-feature spacing on a probe array where the spot size may produce “blurring” in the resulting image. In the present example, the described analysis may preferably be implemented with images generated from a scanning system using a CCD based architecture with a wide field of view which is described in greater detail below.

2. Related Art

Synthesized nucleic acid probe arrays, such as Affymetrix GeneChip® probe arrays, and spotted probe arrays, have been used to generate unprecedented amounts of information about biological systems. For example, the GeneChip® Human Genome U133 Plus 2.0 Array for expression applications available from Affymetrix, Inc. of Santa Clara, Calif., is comprised of one microarray containing 1,300,000 oligonucleotide features covering more than 47,000 transcripts and variants that include 38,500 well characterized human genes. Similarly, the GeneChip® Mapping 500K Array Set for genotyping applications available from Affymetrix, Inc. of Santa Clara, Calif., is comprised of two arrays, each capable of genotyping on average 250,000 SNPs. Analysis of expression or genotyping data from such microarrays may lead to the development of new drugs and new diagnostic tools.

### SUMMARY OF THE INVENTION

Systems, methods, and products to address these and other needs are described herein with respect to illustrative, non-limiting, implementations. Various alternatives, modifications and equivalents are possible. For example, certain systems, methods, and computer software products are described herein using exemplary implementations for analyzing data from arrays of biological materials produced by the Affymetrix® 417™ or 427™ Arrayer. Other illustrative implementations are referred to in relation to data from Affymetrix® GeneChip® probe arrays. However, these systems, methods, and products may be applied with respect to many other types of probe arrays and, more generally, with respect to numerous parallel biological assays produced in accordance with other conventional technologies and/or produced in accordance with techniques that may be developed in the future. For example, the systems, methods, and products described herein may be applied to parallel assays of nucleic acids, PCR products generated from cDNA clones, proteins, antibodies, or many other biological materials. These materials may be disposed on slides (as typically used for spotted arrays), on substrates employed for GeneChip® arrays, or on beads, optical fibers, or other substrates or media, which may include polymeric coatings or other layers on top of slides or other substrates. Moreover, the probes need not be immobilized in or on a substrate, and, if immobilized, need not be disposed in regular patterns or arrays. For convenience, the term “probe array” will generally be used broadly hereafter to refer to all of these types of arrays and parallel biological assays.

In one embodiment, a method of reconstructing an image of a biological probe array is described that comprises the steps of (a) receiving a raw image of a biological probe array comprising a plurality of cells that represents probe features on the probe array, where each cell also comprises a plurality of pixels each comprising a raw intensity value; (b) assigning an intensity value to each of a plurality of reconstructed cells of a reconstructed image, where each reconstructed cell comprises a plurality of reconstructed pixels; (c) determining a weighted intensity value for each reconstructed pixel in each reconstructed cell using the intensity value of the reconstructed cell and a weight value; (d) determining an error value for each reconstructed pixel using the weighted intensity value and the raw intensity value of a corresponding pixel in the raw image; (e) updating the intensity value of each of the reconstructed cells using the error value; and (f) repeating steps (c)-(e) until convergence, where the intensity value for the reconstructed cells of the converged reconstructed image is representative of light emitted from the corresponding probe features.

Also, an implementation a method of reconstructing a cell using a raw image of a biological probe array is described that comprises (a) assigning an intensity value to a reconstructed cell of a reconstructed image, where each reconstructed cell comprises a plurality of reconstructed pixels; (b) determining a weighted intensity value for each reconstructed pixel in the reconstructed cell using the intensity value of the reconstructed cell and a weight value; (c) determining an error value for each reconstructed pixel using the weighted intensity value and a raw intensity value corresponding to a pixel in the raw image; (d) updating the intensity value of the reconstructed cell using the error value; and (e) repeating steps (b)-(d) until convergence, wherein the intensity value for the reconstructed cell is representative of light emitted from a corresponding probe feature on the biological probe array.

The above embodiments and implementations are not necessarily inclusive or exclusive of each other and may be combined in any manner that is non-conflicting and otherwise possible, whether they be presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures or method steps and the leftmost digit of a reference numeral indicates the number of the figure in which the referenced element first appears (for example, the element 160 appears first in FIG. 1). In functional block diagrams, rectangles generally indicate functional elements and parallelograms generally indicate data. In method flow charts, rectangles generally indicate method steps and diamond shapes generally indicate decision elements. All of these conventions, however, are intended to be typical or illustrative, rather than limiting.

FIG. 1 is a functional block diagram of one embodiment of a scanner instrument enabled to scan a probe array and computer system for image acquisition and analysis;

FIG. 2 is a functional block diagram of one embodiment of the scanner-computer system of FIG. 1, including a cartridge transport frame, scanner optics and detectors, and a scanner computer comprising instrument control and image analysis applications;

FIG. 3 is a simplified graphical representation of the scanner optics and detectors of FIG. 2, suitable for providing excitation light and the detection of emission signals;

FIG. 4 is a functional block diagram of one embodiment of the scanner computer of FIG. 3, including a sensor board;

FIG. 5 is a functional block diagram of one embodiment of the instrument control and image analysis applications of FIG. 2;

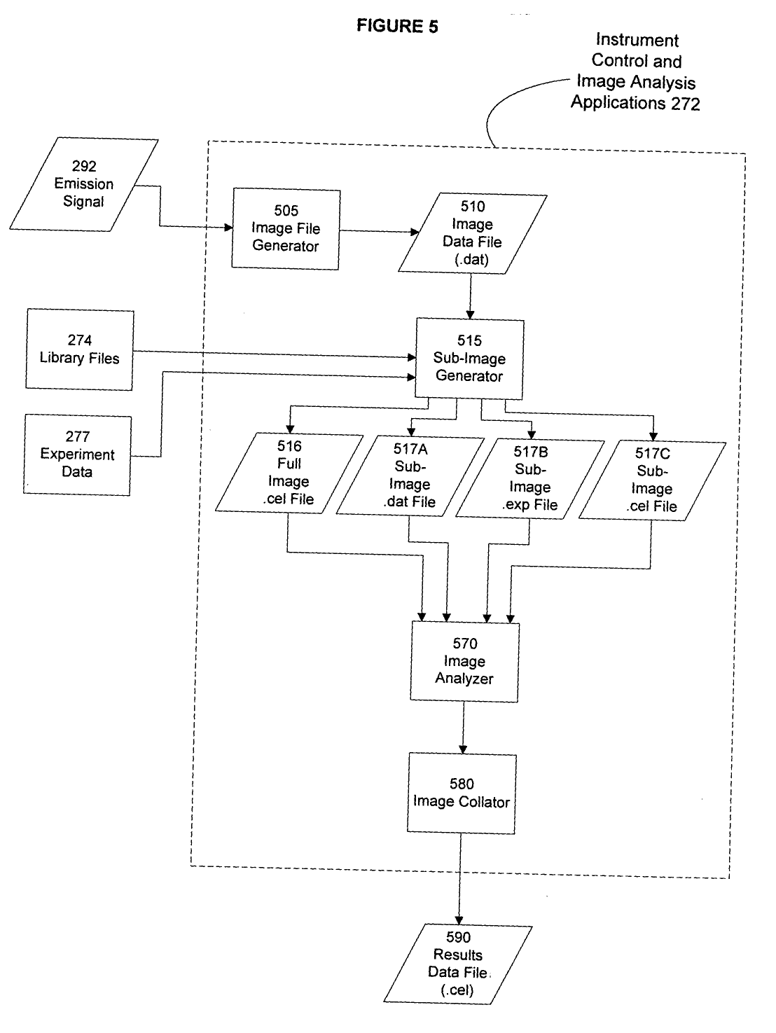

FIG. 6 is a functional block diagram of one embodiment of a method for reconstructing an image employed by the image analysis applications of FIG. 5; and

FIG. 7 is a simplified graphical representation of one embodiment of a graphical plot employed for determining registration accuracy.

### DETAILED DESCRIPTION

### a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841; WO 00/58516; U.S. Pat. Nos. 5,143,854; 5,242,974; 5,252,743; 5,324,633; 5,384,261; 5,405,783; 5,424,186; 5,451,683; 5,482,867; 5,491,074; 5,527,681; 5,550,215; 5,571,639; 5,578,832; 5,593,839; 5,599,695; 5,624,711; 5,631,734; 5,795,716; 5,831,070; 5,837,832; 5,856,101; 5,858,659; 5,936,324; 5,968,740; 5,974,164; 5,981,185; 5,981,956; 6,025,601; 6,033,860; 6,040,193; 6,090,555; 6,136,269; 6,269,846; and 6,428,752; in PCT Applications Nos. PCT/US99/00730 (International Publication No. WO 99/36760); and PCT/US01/04285 (International Publication No. WO 01/58593); which are all incorporated herein by reference in their entirety for all purposes.

Patents that describe synthesis techniques in specific embodiments include U.S. Pat. Nos. 5,412,087; 6,147,205; 6,262,216; 6,310,189; 5,889,165; and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992; 6,013,449; 6,020,135; 6,033,860; 6,040,138; 6,177,248; and 6,309,822. Genotyping and uses therefore are shown in U.S. Ser. Nos. 10/442,021; 10/013,598 (U.S. Patent Application Publication 20030036069); and U.S. Pat. Nos. 5,856,092; 6,300,063; 5,858,659; 6,284,460; 6,361,947; 6,368,799; and 6,333,179. Other uses are embodied in U.S. Pat. Nos. 5,871,928; 5,902,723; 6,045,996; 5,541,061; and 6,197,506.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202; 4,683,195; 4,800,159; 4,965,188; and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909; 5,861,245) and nucleic acid based sequence amplification (NABSA). (See, U.S. Pat. Nos. 5,409,818; 5,554,517; and 6,063,603, each of which is incorporated herein by reference). Other amplification methods that may be used are described in, U.S. Pat. Nos. 5,242,794; 5,494,810; 4,988,617; and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135; 09/920,491 (U.S. Patent Application Publication 20030096235); Ser. No. 09/910,292 (U.S. Patent Application Publication 20030082543); and Ser. No. 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928; 5,874,219; 6,045,996; 6,386,749; and 6,391,623 each of which are incorporated herein by reference.

The present invention also contemplates signal detection of hybridization between ligands in certain preferred embodiments. For example, methods and apparatus for signal detection and processing of intensity data are disclosed in, U.S. Pat. Nos. 5,143,854; 5,547,839; 5,578,832; 5,631,734; 5,800,992; 5,834,758; 5,856,092; 5,902,723; 5,936,324; 5,981,956; 6,025,601; 6,090,555; 6,141,096; 6,171,793; 6,185,030; 6,201,639; 6,207,960; 6,218,803; 6,225,625; 6,252,236; 6,335,824; 6,403,320; 6,407,858; 6,472,671; 6,490,533; 6,650,411; and 6,643,015, in U.S. patent application Ser. Nos. 10/389,194; 60/493,495; and in PCT Application PCT/US99/06097 (published as WO99/47964), each of which also is hereby incorporated by reference in its entirety for all purposes. The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001). See U.S. Pat. No. 6,420,108.

The present invention may also make use of various computer program products and software for a variety of purposes, such as probe design, management of data, analysis, and instrument operation. See, U.S. Pat. Nos. 5,733,729; 5,593,839; 5,795,716; 5,733,729; 5,974,164; 6,066,454; 6,090,555; 6,185,561; 6,188,783; 6,223,127; 6,228,593; 6,229,911; 6,242,180; 6,308,170; 6,361,937; 6,420,108; 6,484,183; 6,505,125; 6,510,391; 6,532,462; 6,546,340; and 6,687,692.

Additionally, the present invention may have preferred embodiments that include methods for providing genetic information over networks such as the Internet as shown in U.S. Ser. Nos. 10/197,621; 10/063,559 (United States Publication Number 20020183936); Ser. Nos. 10/065,856; 10/065,868; 10/328,818; 10/328,872; 10/423,403; and 60/482,389.

### b) Definitions

The term “admixture” refers to the phenomenon of gene flow between populations resulting from migration. Admixture can create linkage disequilibrium (LD).

The term “allele’ as used herein is any one of a number of alternative forms a given locus (position) on a chromosome. An allele may be used to indicate one form of a polymorphism, for example, a biallelic SNP may have possible alleles A and B. An allele may also be used to indicate a particular combination of alleles of two or more SNPs in a given gene or chromosomal segment. The frequency of an allele in a population is the number of times that specific allele appears divided by the total number of alleles of that locus.

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

The term “biopolymer” or sometimes refer by “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

The term “biopolymer synthesis” as used herein is intended to encompass the synthetic production, both organic and inorganic, of a biopolymer. Related to a bioploymer is a “biomonomer”.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “effective amount” as used herein refers to an amount sufficient to induce a desired result.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “genotype” as used herein refers to the genetic information an individual carries at one or more positions in the genome. A genotype may refer to the information present at a single polymorphism, for example, a single SNP. For example, if a SNP is biallelic and can be either an A or a C then if an individual is homozygous for A at that position the genotype of the SNP is homozygous A or AA. Genotype may also refer to the information present at a plurality of polymorphic positions.

The term “Hardy-Weinberg equilibrium” (HWE) as used herein refers to the principle that an allele that when homozygous leads to a disorder that prevents the individual from reproducing does not disappear from the population but remains present in a population in the undetectable heterozygous state at a constant allele frequency.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than about 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations or conditions of 100 mM MES, 1 M [Na+], 20 mM EDTA, 0.01% Tween-20 and a temperature of 30-50° C., preferably at about 45-50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GeneChip Mapping Assay Manual, 2004.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), LNAs, as described in Koshkin et al. Tetrahedron 54:3607-3630, 1998, and US Pat. No. 6,268,490, aptamers, and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture (for example, total cellular) DNA or RNA.

The term “initiation biomonomer” or “initiator biomonomer” as used herein is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

The term “linkage analysis” as used herein refers to a method of genetic analysis in which data are collected from affected families, and regions of the genome are identified that co-segregated with the disease in many independent families or over many generations of an extended pedigree. A disease locus may be identified because it lies in a region of the genome that is shared by all affected members of a pedigree.

The term “linkage disequilibrium” or sometimes referred to as “allelic association” as used herein refers to the preferential association of a particular allele or genetic marker with a specific allele, or genetic marker at a nearby chromosomal location more frequently than expected by chance for any particular allele frequency in the population. For example, if locus X has alleles A and B, which occur equally frequently, and linked locus Y has alleles C and D, which occur equally frequently, one would expect the combination AC to occur with a frequency of 0.25. If AC occurs more frequently, then alleles A and C are in linkage disequilibrium. Linkage disequilibrium may result from natural selection of certain combination of alleles or because an allele has been introduced into a population too recently to have reached equilibrium with linked alleles. The genetic interval around a disease locus may be narrowed by detecting disequilibrium between nearby markers and the disease locus. For additional information on linkage disequilibrium see Ardlie et al., Nat. Rev. Gen. 3:299-309, 2002.

The term “mendelian inheritance” as used herein refers to

The term “lod score” or “LOD” is the log of the odds ratio of the probability of the data occurring under the specific hypothesis relative to the null hypothesis. LOD=log [probability assuming linkage/probability assuming no linkage].

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

The term “monomer” as used herein refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” or sometimes refer by “array” as used herein refers to an intentionally created collection of nucleic acids which can be prepared either synthetically or biosynthetically and screened for biological activity in a variety of different formats (for example, libraries of soluble molecules; and libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, Principles of Biochemistry, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “polymorphism” as used herein refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

### c) EMBODIMENTS OF THE PRESENT INVENTION

Embodiments of an image analysis system are described herein that are enabled to provide reliable data from scanned images of probe arrays comprising small feature sizes. In particular, embodiments are described that are enabled to accurately image and analyze the data associated with features of a probe array that may include feature sizes in a range of 8 μm to 5 μm, 1 μm, or smaller in a dimension (such as the side of a square, side of a rectangle, or diameter of a spot).

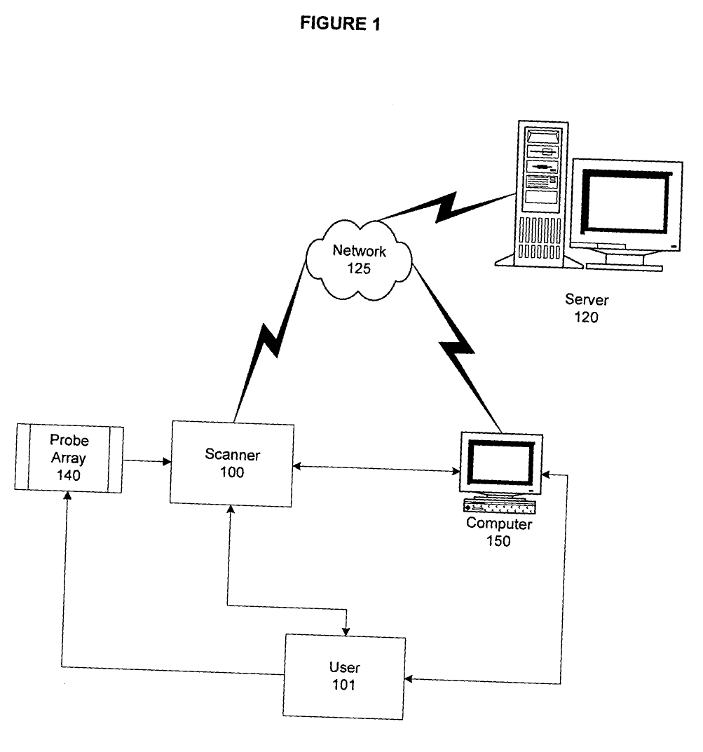

Probe Array 240: An illustrative example of probe array 240 is provided in FIGS. 1, 2, and 3. Descriptions of probe arrays are provided above with respect to “Nucleic Acid Probe arrays” and other related disclosure. In various implementations, probe array 240 may be disposed in a cartridge or housing such as, for example, the GeneChip® probe array available from Affymetrix, Inc. of Santa Clara Calif. Examples of probe arrays and associated cartridges or housings may be found in U.S. Pat. Nos. 5,945,334, 6,287,850, 6,399,365, 6,551,817, each of which is also hereby incorporated by reference herein in its entirety for all purposes. In addition, some embodiments of probe array 240 may be associated with pegs or posts, where for instance probe array 240 may be affixed via gluing, welding, or other means known in the related art to the peg or post that may be operatively coupled to a tray, strip or other type of similar substrate. Examples with embodiments of probe array 240 associated with pegs or posts may be found in U.S. patent Ser. No. 10/826,577, titled “Immersion Array Plates for Interchangeable Microtiter Well Plates”, filed Apr. 16, 2004, which is hereby incorporated by reference herein in its entirety for all purposes.

Server 120: FIG. 1 shows a typical configuration of a server computer connected to a workstation computer via a network. In some implementations any function ascribed to Server 120 may be carried out by one or more other computers, and/or the functions may be performed in parallel by a group of computers. Network 125 may include a local area network, a wide area network, the Internet, another network, or any combination thereof.

Typically, server 120 is a network-server class of computer designed for servicing a number of workstations or other computer platforms over a network. However, server 120 may be any of a variety of types of general-purpose computers such as a personal computer, workstation, main frame computer, or other computer platform now or later developed. Server 120 typically includes known components such as a processor, an operating system, a system memory, memory storage devices, and input-output controllers. It will be understood by those skilled in the relevant art that there are many possible configurations of the components of server 120 that may typically include cache memory, a data backup unit, and many other devices. Similarly, many hardware and associated software or firmware components may be implemented in a network server. For example, components to implement one or more firewalls to protect data and applications, uninterruptable power supplies, LAN switches, web-server routing software, and many other components. Those of ordinary skill in the art will readily appreciate how these and other conventional components may be implemented.

Server 120 may employ one or more processing elements that may, for instance, include multiple processors; e.g., multiple Intel® Xeon™ 3.2 GHz processors. As further examples, the processing elements may include one or more of a variety of other commercially available processors such as Itanium® 2 64-bit processors or Pentium® processors from Intel, SPARC® processors made by Sun Microsystems, Opteron™ processors from Advanced Micro Devices, or other processors that are or will become available. The processing elements execute the operating system, which may be, for example, a Windows®-type operating system (such as Windows Server System that may include Windows Server 2003, SQL Server® 2005, Windows® 2000 with SP 1, Windows NT® 4.0 with SP6a) from the Microsoft Corporation; the Solaris operating system from Sun Microsystems, the Tru64 Unix from Compaq, other Unix® or Linux-type operating systems available from many vendors or open sources; another or a future operating system; or some combination thereof. The operating system interfaces with firmware and hardware in a well-known manner, and facilitates the processor in coordinating and executing the functions of various computer programs that may be written in a variety of programming languages. The operating system, typically in cooperation with the processor, coordinates and executes functions of the other components of server 120. The operating system also provides scheduling, input-output control, file and data management, memory management, and communication control and related services, all in accordance with known techniques.

The system memory may be any of a variety of known or future memory storage devices. Examples include any commonly available random access memory (RAM), magnetic medium such as a resident hard disk or tape, an optical medium such as a read and write compact disc, or other memory storage device. The memory storage device may be any of a variety of known or future devices, including a compact disk drive, a tape drive, a removable hard disk drive, flash memory, or a diskette drive. Such types of memory storage device typically read from, and/or write to, a program storage medium (not shown) such as, respectively, a compact disk, magnetic tape, removable hard disk, flash memory, or floppy diskette. Any of these program storage media, or others now in use or that may later be developed, may be considered a computer program product. As will be appreciated, these program storage media typically store a computer software program and/or data. Computer software programs, also called computer control logic, typically are stored in the system memory and/or the program storage device used in conjunction with the memory storage device.

In some embodiments, a computer program product is described comprising a computer usable medium having control logic (computer software program, including program code) stored therein. The control logic, when executed by the processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

The input-output controllers could include any of a variety of known devices for accepting and processing information from a user, whether a human or a machine, whether local or remote. Such devices include, for example, modem cards, network interface cards, sound cards, or other types of controllers for any of a variety of known input or output devices. In the illustrated embodiment, the functional elements of server 120 communicate with each other via a system bus. Some of these communications may be accomplished in alternative embodiments using network or other types of remote communications.

As will be evident to those skilled in the relevant art, a server application if implemented in software, may be loaded into the system memory and/or the memory storage device through one of the input devices. All or portions of these loaded elements may also reside in a read-only memory or similar device of the memory storage device, such devices not requiring that the elements first be loaded through the input devices. It will be understood by those skilled in the relevant art that any of the loaded elements, or portions of them, may be loaded by the processor in a known manner into the system memory, or cache memory (not shown), or both, as advantageous for execution.

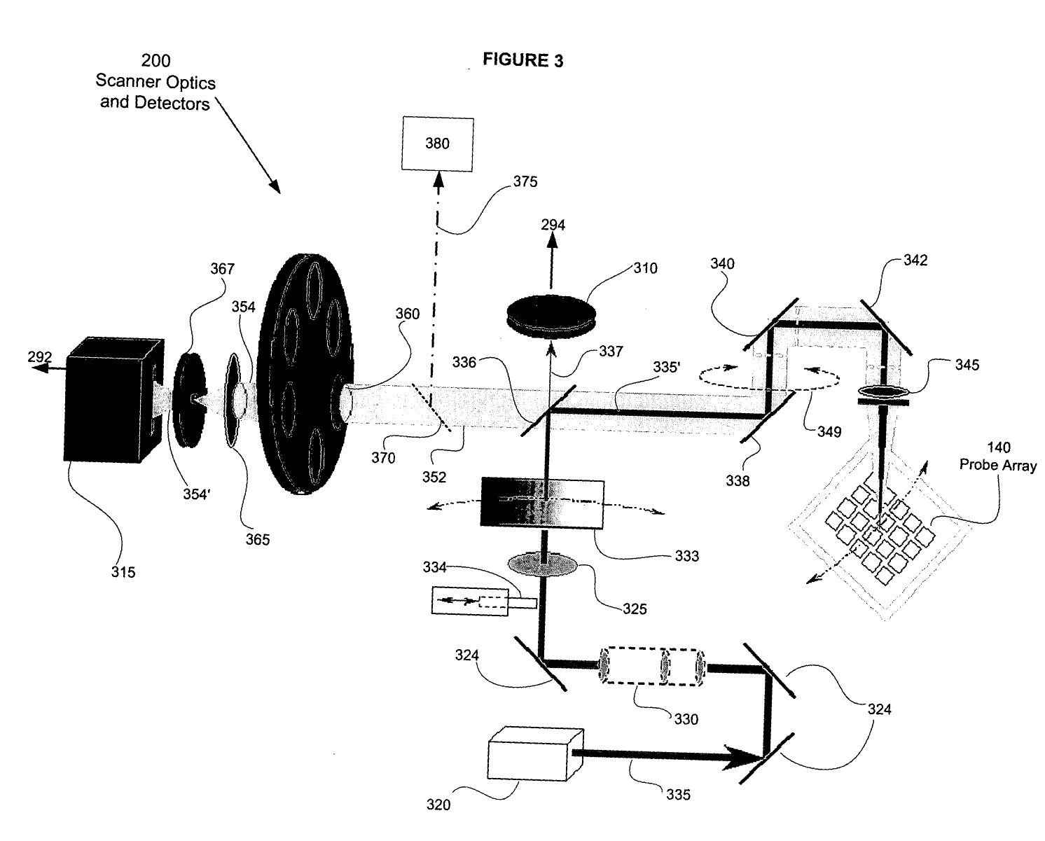

Scanner 100: Labeled targets hybridized to probe arrays may be detected using various devices, sometimes referred to as scanners, as described above with respect to methods and apparatus for signal detection. An illustrative device is shown in FIG. 1 as scanner 100, that may incorporate a variety of optical elements such as the example illustrated in FIG. 3 that includes a plurality of optical elements associated with scanner optics and detectors 200. For example, scanners image the targets by detecting fluorescent or other emissions from labels associated with target molecules, or by detecting transmitted, reflected, or scattered radiation. A typical scheme employs optical and other elements to provide excitation light and to selectively collect the emissions.

For example, scanner 100 provides a signal representing the intensities (and possibly other characteristics, such as color that may be associated with a detected wavelength) of the detected emissions or reflected wavelengths of light, as well as the locations on the substrate where the emissions or reflected wavelengths were detected. Typically, the signal includes intensity information corresponding to elemental sub-areas of the scanned substrate. The term “elemental” in this context means that the intensities, and/or other characteristics, of the emissions or reflected wavelengths from this area each are represented by a single value. When displayed as an image for viewing or processing, elemental picture elements, or pixels, often represent this information. Thus, in the present example, a pixel may have a single value representing the intensity of the elemental sub-area of the substrate from which the emissions or reflected wavelengths were scanned. The pixel may also have another value representing another characteristic, such as color, positive or negative image, or other type of image representation. The size of a pixel may vary in different embodiments and could include a 2.5 μm, 1.5 μm, 1.0 μm, or sub-micron pixel size. Two examples where the signal may be incorporated into data are data files in the form *.dat or *.tif as generated respectively by Affymetrix® Microarray Suite (described in U.S. patent application Ser. No. 10/219,882, which is hereby incorporated by reference herein in its entirety for all purposes) or Affymetrix® GeneChip® Operating Software (described in U.S. patent application Ser. No. 10/764,663, which is hereby incorporated by reference herein in its entirety for all purposes ) based on images scanned from GeneChip® arrays, and Affymetrix® Jaguar™ software (described in U.S. patent application Ser. No. 09/682,071, which is hereby incorporated by reference herein in its entirety for all purposes) based on images scanned from spotted arrays. Examples of scanner systems that may be implemented with embodiments of the present invention include U.S. patent application Ser. Nos. 10/389,194; and 10/913,102, both of which are incorporated by reference above; and U.S. patent application Ser. No. 10/846,261, titled “System, Method, and Product for Providing A Wavelength-Tunable Excitation Beam”, filed May 13, 2004; and U.S. patent application Ser. No. 11/260,617, titled “System, Method and Product for Multiple Wavelength Detection Using Single Source Excitation”, filed Oct. 27, 2005, each of which is hereby incorporated by reference herein in its entirety for all purposes.

Embodiments of the presently described invention may be employed with images generated by implementations of scanner 100 comprising various optical architectures, but may be preferably employed with images generated using an implementation of scanner 100 comprising a CCD based optical architecture with what may be referred to as a wide field of view. For example, a CCD based architecture may employ some or all of the components described with respect to scanner optics and detectors 200, but typically may not need particular components such as, for instance, implementations of pinhole 367 or embodiments of detectors 310 and 315 that include photomultiplier tubes which may be more amenable to a confocal or other similar type of optical architecture.

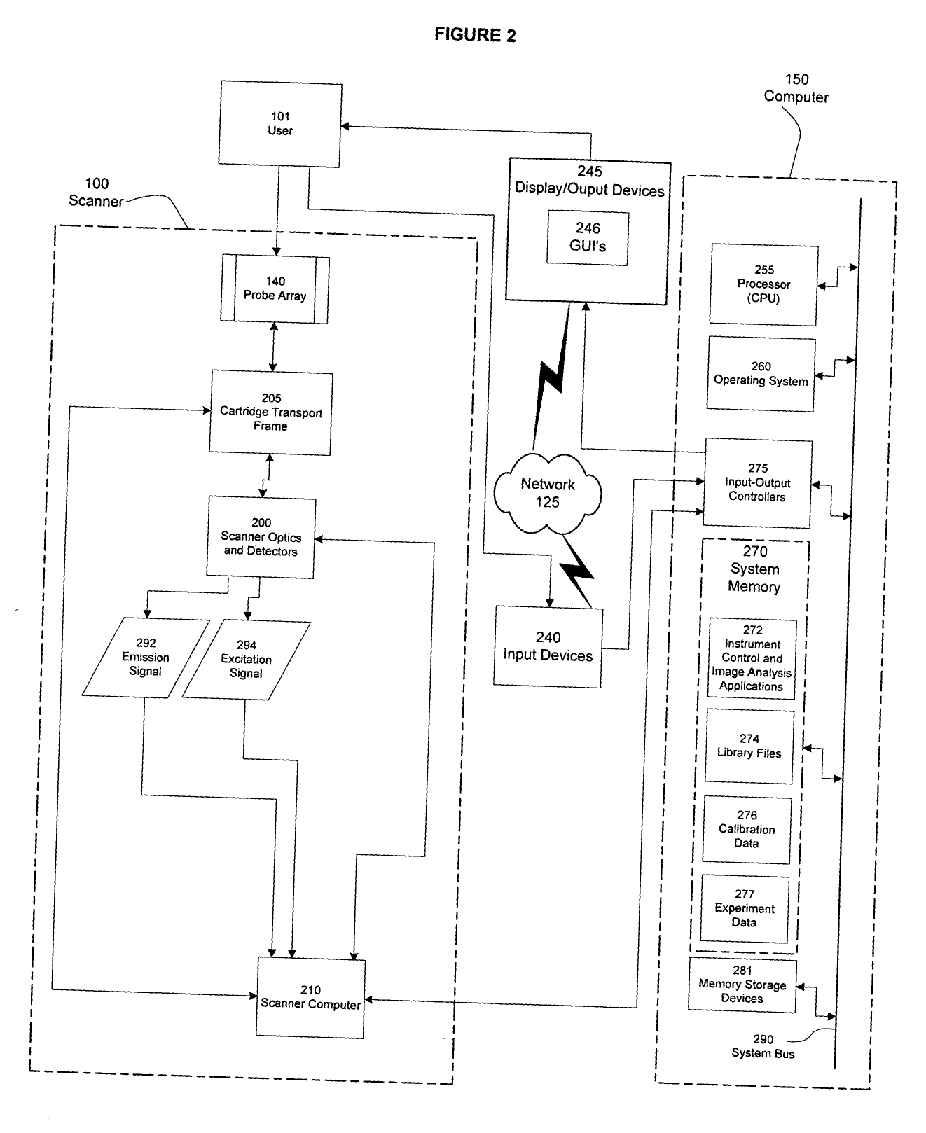

Computer 150: An illustrative example of computer 150 is provided in FIG. 1 and also in greater detail in FIG. 2. Computer 150 may be any type of computer platform such as a workstation, a personal computer, a server, or any other present or future computer. Computer 150 typically includes known components such as a processor 255, an operating system 260, system memory 270, memory storage devices 281, and input-output controllers 275, input devices 240, and display/output devices 245. Display/Output Devices 245 may include display devices that provides visual information, this information typically may be logically and/or physically organized as an array of pixels. A Graphical user interface (GUI) controller may also be included that may comprise any of a variety of known or future software programs for providing graphical input and output interfaces such as for instance GUI's 246. For example, GUI's 246 may provide one or more graphical representations to a user, such as user 101, and also be enabled to process user inputs via GUI's 246 using means of selection or input known to those of ordinary skill in the related art.

It will be understood by those of ordinary skill in the relevant art that there are many possible configurations of the components of computer 150 and that some components that may typically be included in computer 150 are not shown, such as cache memory, a data backup unit, and many other devices. Processor 255 may be a commercially available processor such as an Itanium® or Pentium® processor made by Intel Corporation, a SPARC® processor made by Sun Microsystems, an Athalon™ or Opteron™ processor made by AMD corporation, or it may be one of other processors that are or will become available. Processor 255 executes operating system 260, which may be, for example, a Windows®-type operating system (such as Windows NT® 4.0 with SP6a, or Windows XP) from the Microsoft Corporation; a Unix® or Linux-type operating system available from many vendors or what is referred to as an open source; another or a future operating system; or some combination thereof. Operating system 260 interfaces with firmware and hardware in a well-known manner, and facilitates processor 255 in coordinating and executing the functions of various computer programs that may be written in a variety of programming languages. Operating system 260, typically in cooperation with processor 255, coordinates and executes functions of the other components of computer 150. Operating system 260 also provides scheduling, input-output control, file and data management, memory management, and communication control and related services, all in accordance with known techniques.

System memory 270 may be any of a variety of known or future memory storage devices. Examples include any commonly available random access memory (RAM), magnetic medium such as a resident hard disk or tape, an optical medium such as a read and write compact disc, or other memory storage device. Memory storage devices 281 may be any of a variety of known or future devices, including a compact disk drive, a tape drive, a removable hard disk drive, flash memory, or a diskette drive. Such types of memory storage devices 281 typically read from, and/or write to, a program storage medium (not shown) such as, respectively, a compact disk, magnetic tape, removable hard disk, flash memory, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory 270 and/or the program storage device used in conjunction with memory storage device 281.

In some embodiments, a computer program product is described comprising a computer usable medium having control logic (computer software program, including program code) stored therein. The control logic, when executed by processor 255, causes processor 255 to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers 275 could include any of a variety of known devices for accepting and processing information from a user, whether a human or a machine, whether local or remote. Such devices include, for example, modem cards, network interface cards, sound cards, or other types of controllers for any of a variety of known input devices. Output controllers of input-output controllers 275 could include controllers for any of a variety of known display devices for presenting information to a user, whether a human or a machine, whether local or remote. In the illustrated embodiment, the functional elements of computer 150 communicate with each other via system bus 290. Some of these communications may be accomplished in alternative embodiments using network or other types of remote communications.

As will be evident to those skilled in the relevant art, instrument control and image processing applications 272, if implemented in software, may be loaded into and executed from system memory 270 and/or memory storage device 281. All or portions of applications 272 may also reside in a read-only memory or similar device of memory storage device 281, such devices not requiring that applications 272 first be loaded through input-output controllers 275. It will be understood by those skilled in the relevant art that applications 272, or portions of it, may be loaded by processor 255 in a known manner into system memory 270, or cache memory (not shown), or both, as advantageous for execution. Also illustrated in FIG. 2 are library files 274, calibration data 276, and experiment data 277 stored in system memory 270. For example, calibration data 276 could include one or more values or other types of calibration data related to the calibration of scanner 100 or other instrument. Additionally, experiment data 277 could include data related to one or more experiments or assays such as excitation wavelength ranges, emission wavelength ranges, extinction coefficients and/or associated excitation power level values, or other values associated with one or more fluorescent labels.

Network 125 may include one or more of the many various types of networks well known to those of ordinary skill in the art. For example, network 125 may include what is commonly referred to as a TCP/IP network, or other type of network that may include the internet, or intranet architectures.

Scanner Optics and Detectors 200: FIG. 3 provides a simplified graphical example of possible embodiments of optical elements associated with scanner 100, illustrated as scanner optics and detectors 200. For example, an element of the presently described invention includes source 320 that could comprise one or more Light emitting Diodes (sometimes referred to as LED's), or a wide spectrum light source. For instance, some embodiments of LED's provide sufficient levels of excitation light to evoke fluorescent emissions from fluorophores, where a single LED may be employed as source 320. LED's of this type provide advantages in certain embodiments over other types of sources due to their low cost, high output efficiency, long life, short on/off-off/on transition time, large selection of wavelengths, and low heat production.

Additionally, some implementations could include source 320 that comprises a laser such as, for instance, a solid state, diode pumped, frequency doubled Nd: YAG (Neodymium-doped Yttrium Aluminum Garnet) or YVO4 laser producing green laser light, having a wavelength of 532 nm or other laser implementation. In the present example, source 320 provides light within the excitation range of one or more fluorescent labels associated with target molecules hybridized to probes disposed on probe array 140 or fluorescent labels associated with a calibration standard. Also in the present example, the wavelength of the excitation light provided by source 320 may be tunable such to enable the use multiple color assays (i.e. employing multiple fluorescent labels with distinct ranges of excitation and emission wavelengths) associated with an embodiment of probe array 140 (Further examples of tunable sources are described in U.S. patent application Ser. No. 10/846,261, titled “System, Method, and Product for Providing a Wavelength-Tunable Excitation Beam, filed May 13, 2004, which is hereby incorporated by reference herein in its entirety for all purposes). Those of ordinary skill in the related art will appreciate that other types of sources 320 may be employed in the present invention such as incandescent sources, halogen or xenon sources, metal halide sources, mercury vapor sources, or other sources known in the art.

In some embodiments, a single implementation of source 320 is employed that produces a single excitation beam, illustrated in FIG. 3 as excitation beam 335. Alternative embodiments may include multiple implementations of source 320 that each provide excitation light that may be combined into a single beam or directed along separate optical paths to a target, although those of ordinary skill in the related art will appreciate that there are several advantages to implementing a single source over multiple sources such as complexity, space, power, and expense.

FIG. 3 further provides an illustrative example of the paths of excitation beam 335 and emission beam 352 and a plurality of optical components that comprise scanner optics 200. In the present example, excitation beam 335 is emitted from source 320 and is directed along an optical path by one or more turning mirrors 324 toward a three-lens beam conditioner/expander 330. Turning mirrors are commonly associated with optical systems to provide the necessary adjustments to what may be referred to as the optical path such as, for instance, to allow for alignment of excitation beam 335 at lens 345 and to allow for alignment of emission beam 354 at detector 315. For example, turning mirrors 324 also serve to “fold” the optical path into a more compact size & shape to facilitate overall scanner packaging. The number of turning mirrors 324 may vary in different embodiments and may depend on the requirements of the optical path. In some embodiments it may be desirable that excitation beam 335 has a known diameter. Beam conditioner/expander 330 may provide one or more optical elements that adjust a beam diameter to a value that could, for instance, include a diameter of 1.076 mm+± 10%. For example, the one or more optical elements could include a three-lens beam expander that may increase the diameter of excitation beam 335 to a desired value. Alternatively, the one or more optical elements may reduce the diameter of excitation beam 335 to a desired value. Additionally, the one or more optical elements of beam conditioner/expander 330 may further condition one or more properties of excitation beam 335 to provide other desirable characteristics, such as providing what those of ordinary skill in the related art refer to as a plane wavefront to lens 345. Excitation beam 335 with the desirable characteristics may then exit beam conditioner/expander 330 and continue along the optical path that may again be redirected by one or more turning mirrors 324 towards excitation filter 325.

Filter 325 may be used to remove or block light at wavelengths other than excitation wavelengths, and generally need not be included if, for example, source 320 does not produce light at these extraneous wavelengths. However, it may be desirable in some applications to use inexpensive sources and often it is cheaper to filter out-of-mode light than to design the source to avoid producing such extraneous emissions. In some embodiments, filter 325 allows all or a substantial portion of light at one or more excitation wavelengths to pass through without affecting other characteristics of excitation beam 335, such as the desirable characteristics modified by beam conditioner/expander 330. Also, a plurality of filters 325 may also be associated with a filter wheel or other means for selectively translating a desired filter in the optical path.

After exiting filter 325 excitation beam 335 may then be directed along the optical path to attenuator 333. Attenuator 333 may provide a means for adjusting the level of power of excitation beam 335. In some embodiments, attenuator 333 may, for instance, be comprised of a variable neutral density filter. Those of ordinary skill in the related art will appreciate that neutral density filters, such as absorptive, metallic, or other type of neutral density filter, may be used for reducing the amount of light that is allowed to pass through. The amount of light reduction may depend upon what is referred to as the density of the filter, for instance, as the density increases the amount of light allowed through decreases. The neutral density filter may additionally include a density gradient. For example, an embodiment of attenuator 333 may include a neutral density filter with a density gradient. Attenuator 333, acting under the control of applications 272 and/or firmware 472 may use a step motor that alters the position of the neutral density filter with respect to the optical path of beam 335. The neutral density filter thus reduces the amount of light allowed to pass through based, at least in part, upon the position of the filter gradient relative to the optical path of beam 335. In the present example, the power level of excitation beam is measured by laser power monitor 310 that is described further below, and may be dynamically adjusted to a desired level.

Some embodiments may include one or more implementations of shutter 334. Some implementations may include positioning shutter 334 in one or more locations within scanner 100, along the optical path of excitation beam 335 such that shutter 334 provides a means to block all excitation light from reaching probe array 140, and in some implementations additionally blocking all excitation light from reaching power monitor 310. Shutter 334 may use a variety of means to completely block excitation beam 335. For example shutter 334 may use a motor under the control of applications 272 and/or firmware 472 to extend/retract a solid barrier that could be constructed of metal, plastic, or other appropriate material capable of blocking essentially all light from source 320, such as excitation beam 335. Shutter 334 may be used for a variety of purposes such as, for example, for blocking all light from one or more photo detectors or monitors, including detector 315 and power monitor 310. In the present example, blocking the light may be used for calibration methods that measure and make adjustments to what is referred to as the “dark current” or background noise generated from a number of possible sources such as one or more of the photo detectors, electrical interference, or other sources of noise known to those of ordinary skill in the related art.

In some embodiments of scanner optics and detectors 200, one or more components may be placed in the optical path after elements such as attenuator 333 and/or shutter 334 such as, for instance, beam splitter 336. Those of ordinary skill in the related art will appreciate that beam splitter 336 may include a dichroic beam splitter, also commonly referred to as a dichroic mirror, may include an optical element that is highly reflective to light of a certain wavelength range, and allow transmission of light through the beam splitter or mirror at one or more other wavelength ranges. In some embodiments, beam splitter 336 could also include what is referred to as a geometric beam splitter where a portion of the surface of beam splitter 336 is reflective to all light or light within a particular range of wavelengths, and the remaining portion is permissive to the light. Also, some embodiments of beam splitter 336 may reflect a certain percentage of light at a particular wavelength and allow transmission of the remaining percentage. For example, beam splitter 336 may direct most of the excitation beam, illustrated as excitation beam 335′, along an optical path towards lens 345 while allowing a small fractional portion of excitation beam 335 that is not reflected to pass through beam splitter 336, illustrated in FIG. 3 as partial excitation beam 337. In the present example, partial excitation beam 337 passes through beam splitter 336 to power monitor 310 for the purpose of measuring the power level of excitation beam 335 and providing feedback to applications 272 and/ firmware 472. Applications 272 and/or firmware 472 may then make adjustments, if necessary, to the power level via attenuator 333 as described above.

Monitor 310 may be any of a variety of conventional devices for detecting partial excitation beam 337, such as a silicon detector for providing an electrical signal representative of detected light, a photodiode, a charge-coupled device, a photomultiplier tube, or any other detection device for providing a signal indicative of detected light that is now available or that may be developed in the future. As illustrated in FIG. 3, detector 310 generates excitation signal 294 that represents the detected signal from partial excitation beam 337. In accordance with known techniques, the amplitude, phase, or other characteristic of excitation signal 294 is designed to vary in a known or determinable fashion depending on the power of excitation beam 335. The term “power” in this context refers to the capability of beam 335 to evoke emissions. For example, the power of beam 335 generally refers to photon number or energy per unit of time and typically may be measured in milliwatts of light energy with respect to the illustrated example in which the light energy evokes a fluorescent signal. Thus, excitation signal 294 includes values that represent the power of beam 335 during particular times or time periods. Applications 272 and/or firmware 472 may receive signal 294 for evaluation and, as described above, if necessary make adjustments.

After reflection from beam splitter 336, excitation beam 335′ may continue along an optical path that may in some embodiments be directed via periscope mirror 338, turning mirror 340, and arm end turning mirror 342 to objective lens 345. In the illustrated implementation mirrors 338, 340, and 342 may have the same reflective properties as turning mirrors 324, and could, in some implementations, be used interchangeably with turning mirrors 324.

In some embodiments, lens 345 may include what may be referred to as a diffraction limited optical element that in some implementations comprises a small light weight lens. As described above, in a CCD type architecture it may typically be desirable for lens 345 to have what may be referred to as a wide field of view that may for instance comprise characteristics such as what those of ordinary skill in the related art may refer to as an Airy point spread function.

Also, some embodiments of lens 345 may be positioned in a stationary position where probe array 140 may be translated relative to lens 345, or alternatively lens 345 may be positioned such that it is translated relative to probe array 140. For example, lens 345 may be positioned on a gantry or the end of an arm that is driven by a voice coil for linear translation or a galvanometer for translation around an axis perpendicular to the plane represented by galvo rotation 349 that is a plane parallel to the plane of probe array 140 comprising the probes. In some embodiments, lens 345 focuses excitation beam 335′ down to a specified spot size at the plane of focus that could, for instance, include a 3.5 μm, 2.5 μm or smaller spot size. In the presently described example, galvo rotation 349 results in objective lens 345 moving in an arc over a substrate, providing what may be referred to as an arcuate path that may also be referred to herein as a “scanning line”, upon which biological materials typically have been synthesized or have been deposited. The arcuate path may, for instance, move in a 36 degree arc over a substrate. One or more fluorophores associated with the biological materials emit emission beam 352 at characteristic wavelengths in accordance with well-known principles. The term “fluorophore” commonly refers to a molecule which will absorb energy of a specific wavelength and re-emit energy at a different wavelength. Continuing with the present example, excitation beam 335′ may be focused to a spot by lens 345 and translated in a particular axis with respect to probe array 140 thus providing excitation energy to the probe features along that axis. Additional means of translation may also include a voice coil as described above, a rotating mirror, or other means known to those of ordinary skill in the related art.

Emission beam 352 in the illustrated example follows the reverse optical path as described with respect to excitation beam 335′ until reaching beam splitter 336. In accordance with well known techniques and principles, the characteristics of beam splitter 336 are selected so that beam 352 (or a portion of it) passes through the mirror rather than being reflected. Emission beam 352 is then directed along a desired optical path to filter wheel 360.

In one embodiment, filter wheel 360 may be provided to filter out spectral components of emission beam 352 that are outside of the emission spectra of one or more particular fluorophore species. The term “emission spectra” generally refers to one or more characteristic emission wavelengths or range of wavelengths of those fluorophore species that are responsive to excitation beam 335. In some implementations filter wheel 360 is capable of holding a plurality of filters that each could be tuned to different wavelengths corresponding to the emission spectra from different fluorophore species. Filter wheel 360 may include a mechanism for turning the wheel to position a desired filter in the optical path of emission beam 352. The mechanism may include a motor or some other device for turning or translation that may be responsive to instructions from application 272 and/or firmware 472. For example, excitation beam 335 from source 320 may comprise one or more wavelengths that may include a range of wavelengths that excite one or more fluorophore species where the amount of energy absorbed and re-emitted by each fluorophore species in its emission spectra is a function of its extinction coefficient and the power level of beam 335. In the present example, filter wheel 360 may be translated with respect to the optical path of emission beam 352 to position a filter that is complementary to the emission spectra of the particular fluorophore species in order to remove light components from emission beam 352 that are outside of the emission spectra. The source of the undesirable light components could include undesirable fluorescence generated by other fluorophore species, emissions from glass, glue, or other components associated with elements such as supports, substrates, or housings for probe array 140, or other sources known to those of ordinary skill in the related art.

As an additional example, experiments could be carried out on the same implementation of probe array 140 with a plurality of fluorophore species each with different emission spectra in response to a particular wavelength of excitation light . Such fluorescent species could include molecules capable of what is known in the related art as fluorescent resonant energy transfer (FRET), or semiconductor nanocrystals (sometimes referred to as Quantum Dots). Those of ordinary skill in the related art will appreciate that FRET may be achieved when there are two fluorophore species present in the same molecule. The emission wavelength of one fluorophore overlaps the excitation wavelength of the second fluorophore and results in the emission of a wavelength from the second fluorophore that is atypical of the class of fluorophores that use that excitation wavelength. Also, quantum dots are tunable such that multiple quantum dot species may be employed so that each specie excites at a particular wavelength but has a different characteristic emission spectra. Thus by using an excitation beam of a single wavelength it is possible to obtain distinctly different emissions so that different features of a probe array could be labeled in a single experiment. In the present example, filter wheel 360 may include a complementary filter for each fluorophore specie associated with probe array 140. The result may include filtered emission beam 354 that is a representation of emission beam 352 that has been filtered by a desired filter of filter wheel 360.

In other implementations, multiple excitation sources 320 (or one or more adjustable-wavelength excitation sources) and corresponding multiple optical elements in optical paths similar to the illustrated one could be employed for simultaneous scans at multiple wavelengths. Other examples of scanner systems that utilize multiple emission wavelengths are described in U.S. Pat. No. 6,490,533, titled “System, Method, and Product For Dynamic Noise Reduction in Scanning of Biological Materials”, filed Dec. 3, 2001; U.S. Pat. No. 6,650,411, titled “System, Method, and Product for Pixel Clocking in Scanning of Biological Materials”, filed Dec. 3, 2001; and U.S. Pat. No. 6,643,015, titled “System, Method, and Product for Symmetrical Filtering in Scanning of Biological Materials”, filed Dec. 3, 2001 each of which are hereby incorporated by reference in their entireties for all purposes.

In accordance with techniques well known to those of ordinary skill in the relevant arts, including that of confocal microscopy, beam 354 may be focused by various optical elements such as lens 365 and passed through illustrative pinhole 367, aperture, or other element. In accordance with known techniques, pinhole 367 is defined by and comprises an opening or aperture in substrate 368 and is positioned such that it rejects light from focal planes other than the plane of focus of objective lens 345 (i.e., out-of-focus light), and thus increases the resolution of resulting images. Those of ordinary skill in the related art will appreciate that some scanner architectures will not require an implementation of pinhole 367 such as, for instance, non-confocal architectures that may employ CCD or other similar types of detection elements.

In some implementations, pinhole 367 may be bi-directionally moveable along the optical path. As those of ordinary skill in the related art will appreciate, the appropriate placement of pinhole 367 to reject out of focus light is dependant upon the emission spectra of beam 354 and the diameter of pinhole 367. Those of ordinary skill in the related art will appreciate that it is desirable in many embodiments to reduce the diameter of pinhole 367 to a minimum size associated with the desired focal plane in order to reduce the level of “background” noise in the detected signal. Pinhole 367 may be movable via a motor or other means under the control of applications 272 and/or firmware 472 to a position that corresponds to the emission spectra of the fluorophore species being scanned. In the same or alternative embodiments, pinhole 367 may comprise a sufficiently large diameter to accommodate the wavelengths in the emission spectra of several fluorophore species if those wavelengths are relatively similar to each other, although as described above increasing the diameter of the pinhole may have negative consequences. Also, some embodiments of pinhole 367 may include an “iris” type of aperture that expands and contracts so that the diameter of the hole or aperture is sufficient to permit the desired wavelength of light at the plane of focus to pass through while rejecting light that is substantially out of focus.

Alternatively, some embodiments may include a series of pinholes 367. For example, there may be an implementation of pinhole 367 associated with each fluorophore species associated with probe array140. Each implementation of pinhole 367 may be placed in the appropriate position to reject out of focus light corresponding to the emission spectra of its associated fluorophore. Each of pinholes 367 may be mounted on a translatable stage, rotatable axis, or other means to move pinhole 367 in and out of the optical path. In the present example, the implementation of pinhole 367 corresponding to the fluorophore species being scanned is positioned in the optical path under the control of applications 272 or firmware 472, while the other implementations of pinhole 367 are positioned outside of the optical path thus allowing the implementation of pinhole 367 in the optical path to reject out of focus light.

After passing through pinhole 367, the portion of filtered emission beam 354 that corresponds to the plane of focus, represented as filtered emission beam 354′, continues along a desired optical path and impinges upon detector 315.

Similar to excitation detector 310, emission detector 315 may be a silicon detector for providing an electrical signal representative of detected light, or it may be a photodiode, a charge-coupled device (i.e. CCD), a photomultiplier tube, or any other detection device that is now available or that may be developed in the future for providing a signal indicative of detected light. Detector 315 generates signal 292, that may in some embodiments comprise values associated with photon counts or other measure of intensity that represents filtered emission beam 354′ in the manner noted above with respect to the generation of excitation signal 294 by detector 310. Signal 292 and excitation signal 294 may be provided to applications 272 and/or firmware 472 for processing, as previously described.

Transport frame 205: Another element of scanner 100 may, in some embodiments, include transport frame 205 that provides all of the degrees of freedom required to manipulate probe array 140 for the purposes of auto-focus, scanning, and calibration operations. Those of ordinary skill in the related art will appreciate that the term “degrees of freedom” generally refers to the number of independent parameters required to specify the position and orientation of an object. For example, in one embodiment, probe array 140 may be surrounded or encased by a housing that for instance could include a cartridge with a clear window for optical access to probe array 140. In the present example the cartridge could include one or more features such as a tab or keyed element that interfaces with transport frame 205 and defines the positional relationship of frame 205 and the cartridge. Alternatively, embodiments of probe array 140 may be disposed upon a peg or post type of structure that is operatively coupled to a substrate such as a tray or strip, where the embodiments of probe array 140 is spaced apart from the substrate by a distance that is equal to the height of the peg or post. Frame 205 may then manipulate the position of the cartridge or peg/post substrate relative to one or more elements of scanner 100 such as, for instance, lens 345.

In one embodiment, transport frame 205 is capable of manipulating the cartridge in six possible degrees of freedom such as, for example, what may be generally referred to as yaw, roll, pitch, Z, X and Y.

Probe array 140 may be brought into best focus by adjusting the distance between probe array 140 and lens 345. In some implementations, the distance adjustment may be employed by moving the position of one or more elements of transport frame 205, such as a focus stage, in the Z axis For example, movement of the focus stage in the Z axis may be actuated by one or more motors in a first direction that may decrease the distance between probe array 140 and lens 345, as well as the opposite direction that may increase the distance.

Translation of probe array 140 along the X, and Y axes may in one embodiment be accomplished by a precision linear stage, coupled to what is referred to as one or more micro-stepped motor/drivers, open loop drive mechanism or other type of motorized mechanism. The linear stage may include one or more guide elements to support and guide the housing or cartridge and additional elements to secure the housing or cartridge during scanner operation. In some embodiments, the linear stage may include independent position adjustment mechanisms enabled to adjust the position of probe array 140 in a plurality of axes such that adjustment in one axis is less likely to affect the adjustments in other axes.

In some implementations, the housing or substrate generally remains in the same plane of orientation with respect to scanner 100 from the point that it is loaded into scanner 100 to the point at which it is ejected. This may apply to all operations of the scanner including the auto-focus and scan operations. For example, the cartridge may be received by the scanner at the load position in a vertical orientation, where probe array 140 would be located on one of the side faces of the cartridge. While remaining in the same vertical orientation the cartridge is placed into transport frame 205. Probe array 140, housed in the cartridge, is positioned into the best plane of focus by manipulating the cartridge via the pitch, roll, and Z mechanisms. The probe array is then scanned in the X axis by translation of lens 345 as well as the Y axis by translation of transport frame 205. After the completion of the scan operations the cartridge is returned to the load position via transport frame 205 in the same vertical orientation that it was received in.

Additional examples of cartridge transport frames and means for manipulating the position of a probe array for the purposes of scanning are described in U.S. patent application Ser. No. 10/389,194, incorporated by reference above.

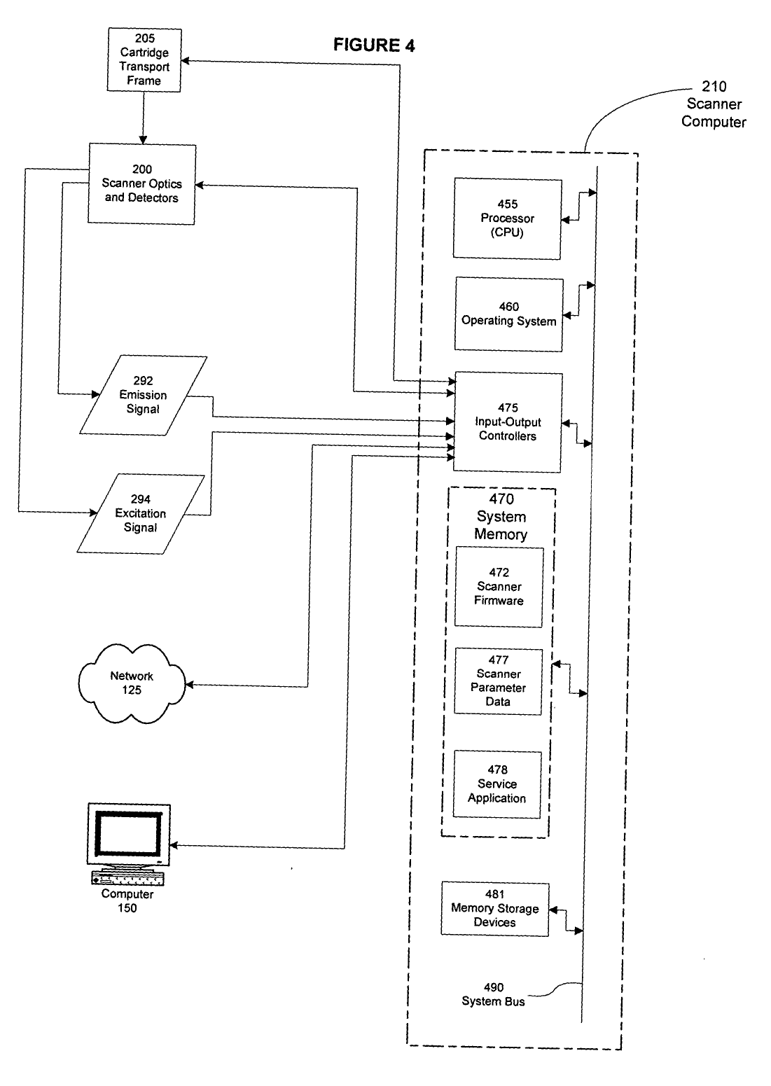

Scanner Computer 210: As illustrated in FIG. 4, scanner computer 210 may include elements such as sensor board 453, processor 455, operating system 460, input-output controllers 475, system memory 470, memory storage devices 481, and system bus 490 that may, in some implementations, have the same characteristics of corresponding elements in computer 150. Other elements of scanner computer 210 may include scanner firmware 472, scanner parameter data 477, and service application 478 that will each be described in detail below.

Scanner firmware 472 may, in many implementations, be enabled to control all functions of scanner 100 based, at least in part, upon data stored locally in scanner parameter data 477 or remotely in one or more data files from one or more remote sources. For example, the remote data source could include computer 150 that includes library files 274, calibration data 276, and experiment data 277 stored in system memory 270. In the present example, the flow of data to scanner computer 210 may be managed by instrument control and image analysis applications 272 that may be responsive to data requests from firmware 472.

A possible advantage of including scanner computer 210 in a particular implementation is that scanner 100 may be network based and/or otherwise arranged so that a user computer, such as computer 150, is not required. Input-output controllers 475 may include what is commonly referred to by those of ordinary skill in the related art as a TCP/IP network connection. The term “TCP/IP” generally refers to a set of protocols that enable the connection of a number of different networks into a network of networks (i.e. the Internet). Scanner computer 210 may use the network connection to connect to one or more computers, such as computer 150, in place of a traditional configuration that includes a “hardwire” connection between a scanner instrument and a single computer. For example, the network connection of input-output controllers 475 may allow for scanner 100 and one more computers to be located remotely from one another. Additionally, a plurality of users, each with their own computer, may utilize scanner 100 independently. In some implementations it is desirable that only a single computer is allowed to connect to scanner 100 at a time. Alternatively, a single computer may interact with a plurality of scanners. In the present example, all calibration and instrument specific information may be stored in one or more locations in scanner computer 210 that may be made available to the one or more computers as they interface with scanner computer 210.

The network based implementation of scanner 100 described above may include methods that enable scanner 100 to operate unimpaired during averse situations that, for instance, may include network disconnects, heavy network loading, electrical interference with the network connection, or other types of adverse event. In some implementations, scanner 100 may require a periodic signal from computer 150 to indicate that the connection is intact. If scanner 100 does not receive that signal within an expected period of time, scanner 100 may operate on the assumption that the network connection has been lost and start storing data that would have been transmitted. When the network connection has been reacquired to scanner 100, all collected data and related information may be transferred to computer 150 that would have normally been transferred if the network connection remained intact. For example, during the occurrence of an adverse situation scanner 100 may lose the network connection to computer 150. The methods enable scanner 100 to operate normally including the acquisition of image data and other operations without interruption. Scanner 100 may store the acquired image data of at least one complete scanned image in memory storage devices 481 to insure that the data is not lost.

In some embodiments, scanner computer 210 may also enable scanner 100 to be configured as a standalone instrument that does not depend upon a controlling workstation. Scanner computer 210 may acquire and store image data as well as function as a data server to multiple clients for efficient data transfer. For example, memory storage devices 481 may include a hard disk or other type of mass storage medium that may be enabled to hold large volumes of image, calibration, and scanner parameter data. Scanner 100 may additionally include a barcode reader, RFID detector, Magnetic strip detector, or other type of device that reads one or more identifiers from one or more labels or tags associated with probe array 140. Scanner computer 210 may execute the scan operations based, at least in part, upon one or more data files associated with the identifiers, and store the acquired image data on the hard disk. Additionally, scanner 100 may provide a network file system or FTP service enabling one or more remote computers to query and upload scanned images as well as providing an interface enabling the computer to query scanner data and statistics.

It will be understood by those of ordinary skill in the related art that the operations of scanner computer 210 may be performed by a variety of other servers or computers, such as for instance computer 150, a server such as a GCOS server, or that computer 210 may not necessarily reside in scanner 100.

Instrument control and image processing applications 272: Instrument control and image processing applications 272 may be any of a variety of known or future image processing applications. Examples of applications 272 include Affymetrix® Microarray Suite, Affymetrix® GeneChip® Operating Software (hereafter referred to as GCOS), and Affymetrix® Jaguar™ software, noted above. Applications 272 may be loaded into system memory 270 and/or memory storage device 281 through one of input devices 240.

Embodiments of applications 272 include executable code being stored in system memory 270 of an implementation of computer 150. Applications 272 may provide a user interface for both the client workstation and one or more servers 120 such as, for instance, GeneChip® Operating Software Server (GCOS Server) available from Affymetrix, Inc. Santa Clara, Calif. Applications 272 could additionally provide the user interface for one or more other workstations and/or one or more instruments. In the presently described implementation, the interface may communicate with and control one or more elements of the one or more servers, one or more workstations, and the one or more instruments. In the described implementation the client workstation could be located locally or remotely to the one or more servers and/or one or more other workstations, and/or one or more instruments. The user interface may, in the present implementation, include an interactive graphical user interface (generally referred to as a GUI), such as GUI's 246, that allow a user to make selections based upon information presented in the GUI. For example, applications 272 may provide an GUI 246 that allows a user to select from a variety of options including data selection, experiment parameters, calibration values, probe array information. Applications 272 may also provide a graphical representation of raw or processed image data where the processed image data may also include annotation information superimposed upon the image such as, for instance, base calls, features of the probe array, or other useful annotation information. Further examples of providing annotation information on image data are provided in U.S. Provisional Patent Application Ser. No. 60/493,950, titled “System, Method, and Product for Displaying Annotation Information Associated with Microarray Image Data”, filed Aug. 8, 2003, which is hereby incorporated by reference herein in its entirety for all purposes.

In alternative implementations, applications 272 may be executed on a server, or on one or more other computer platforms connected directly or indirectly (e.g., via another network, including the Internet or an Intranet) to network 125.

Embodiments of applications 272 also include instrument control features. The instrument control features may include the control of one or more elements of one or more instruments that could, for instance, include elements of a fluid processing station, what may be referred to as an automatic cartridge or tray loader, one or more robotic elements, and scanner 100. The instrument control features may also be capable of receiving information from the one more instruments that could include experiment or instrument status, process steps, or other relevant information. The instrument control features could, for example, be under the control of or an element of the user interface. In the present example, a user may input desired control commands and/or receive the instrument control information via one of GUI's 246. Additional examples of instrument control via a GUI or other interface is provided in U.S. Provisional Patent Application Ser. No. 60/483,812, titled “System, Method and Computer Software for Instrument Control, Data Acquisition and Analysis”, filed Jun. 30, 2003, which is hereby incorporated by reference herein in its entirety for all purposes.

In some embodiments, image data is operated upon by applications 272 to generate intermediate results. Examples of intermediate results include so-called cell intensity files (*.cel) and chip files (*.chp) generated by Affymetrix® GeneChip® Operating Software or Affymetrix® Microarray Suite (as described, for example, in U.S. patent application Ser. Nos. 10/219,882, and 10/764,663, both of which are hereby incorporated herein by reference in their entireties for all purposes) and spot files (*.spt) generated by Affymetrix® Jaguar™ software (as described, for example, in PCT Application PCT/US 01/26390 and in U.S. patent applications Ser. Nos. 09/681,819, 09/682,071, 09/682,074, and 09/682,076, all of which are hereby incorporated by reference herein in their entireties for all purposes). For convenience, the term “file” often is used herein to refer to data generated or used by applications 272 and executable counterparts of other applications, but any of a variety of alternative techniques known in the relevant art for storing, conveying, and/or manipulating data may be employed.

For example, applications 272 receives image data derived from a GeneChip® probe array and generates a cell intensity file. This file contains, for each probe scanned by scanner 100, a single value representative of the intensities of pixels measured by scanner 100 for that probe. Thus, this value is a measure of the abundance of tagged mRNA's present in the target that hybridized to the corresponding probe. Many such mRNA's may be present in each probe, as a probe on a GeneChip® probe array may include, for example, millions of oligonucleotides designed to detect the mRNA's. As noted, another file illustratively assumed to be generated by applications 272 is a chip file. In the present example, in which applications 272 include Affymetrix® GeneChip® Operating Software, the chip file is derived from analysis of the *.cel file combined in some cases with information derived from lab data and/or library files 274 that specify details regarding the sequences and locations of probes and controls. The resulting data stored in the chip file includes degrees of hybridization, absolute and/or differential (over two or more experiments) expression, genotype comparisons, detection of polymorphisms and mutations, and other analytical results.

In another example, in which applications 272 includes Affymetrix® Jaguar™ software operating on image data from a spotted probe array, the resulting spot file includes the intensities of labeled targets that hybridized to probes in the array. Further details regarding cell files, chip files, and spot files are provided in U.S. patent application No. 09/682,074 incorporated by reference above, as well as Ser. Nos. 10/126,468; and 09/682,098; which are hereby incorporated by reference herein in their entireties for all purposes. As will be appreciated by those skilled in the relevant art, the preceding and following descriptions of files generated by applications 272 are exemplary only, and the data described, and other data, may be processed, combined, arranged, and/or presented in many other ways.

User 101 and/or automated data input devices or programs (not shown) may provide data related to the design or conduct of experiments. As one further non-limiting example related to the processing of an Affymetrix® GeneChip® probe array, the user may specify an Affymetrix catalogue or custom chip type (e.g., Human Genome U133 plus 2.0 chip) either by selecting from a predetermined list presented by GCOS or by scanning a bar code, Radio Frequency Identification (RFID), or other means of electronic identification related to a chip to read its type. GCOS may associate the chip type with various scanning parameters stored in data tables including the area of the chip that is to be scanned, the location of chrome borders on the chip used for auto-focusing, the wavelength or intensity/power of excitation light to be used in reading the chip, and so on. As noted, applications 285 may apply some of this data in the generation of intermediate results. For example, information about the dyes may be incorporated into determinations of relative expression.

Those of ordinary skill in the related art will appreciate that one or more operations of applications 272 may be performed by software or firmware associated with various instruments. For example, scanner 100 could include a computer that may include a firmware component that performs or controls one or more operations associated with scanner 100, such as for instance scanner computer 210 and scanner firmware 472.

Some embodiments of applications 272 may be enabled to analyze data produced by scanning implementations of probe array 140 that comprise small feature sizes relative to one or more elements or characteristics of scanner 100 or probe array 140. For example, some embodiments of probe array 140 may comprise 1 million, 6 million, or more probe features, where each probe feature occupies an area of the substrate of probe array 140 that, as those of ordinary skill will appreciate, becomes increasingly small as the density of probe features on probe array 140 increases. Embodiments of probe array 140 may comprise probe features that are square, rectangular, octagonal, hexagonal, round, or other shape where each probe feature may also be separated from each other by a boundary region where there are no probe sequences disposed upon the substrate and in some embodiments may be useful to provide an indicative level of the amount of background signal (i.e. signal not generated by emission from the hybridized probes) in the acquired image. As previously stated, each probe feature may range in size including 8 μm, 5 μm, 1 μm, or smaller in a dimension (such as the side of a square, side of a rectangle, dimension at the widest point, or diameter of a spot), and each boundary region between probe features may be similarly small including a 1 μm, or smaller boundary.

As the probe features of probe array 140 become increasingly small with respect to elements or characteristics of the system, such as scanner 100 and/or applications 272, it may be come increasingly difficult to analyze the data produced in order to make reliable determinations of hybridization events associated with one or more of the probe features. For example, scanner 100 includes source 320 that, as described above, may include a laser, wide spectrum bulb, LED, or other source, that produces excitation light that is focused by lens 345 to a spot, where the dimension of the spot may overlap with a plurality of probe features. The dimension of the spot may vary as described above, and in certain embodiments may be dependent upon characteristics of lens 345 and the distance of probe array 140 from lens 345, where the preferred focus distance may include a point where the dimension of the spot is at its smallest such as for instance a focused spot with a 2.5 μm diameter. In the present example, a 2.5 μm spot is large relative to an 8 μm, 5 μm, 1 μm, or smaller probe feature size, and further each pixel of the resulting image may also include a small dimension such as, for instance, a pixel of 1 μm, 0.7 μm, 0.5 μm or smaller in dimension and thus each probe feature may comprise a plurality of pixels.

Those of ordinary skill in the related art will appreciate that as the spot is exposed to probe array 140 a proportion of the spot comprising the center of the spot may cover a first probe feature and the remaining proportion of the spot may overlap into the boundary area and one or more other neighboring probe features. Therefore, assigning an intensity value to the probe feature associated with the center of the spot using light collected from the entire spot dimension may influenced by the intensity detected from the boundary area as well as the one or more neighboring probe features creating error.

For example, some embodiments of applications 272 may analyze and “reconstruct” the data from a raw acquired image of probe array 140 in order to extract the intensity information associated with each probe feature and produce an image the reliably represents the intensity of each probe feature. In the present example, the raw image may include image data file (.dat) 510 generated by image file generator 505 using emission signal 292 produced by scanner 100 that scans probe array 140

Sub Image Generator 515: In some implementations, sub-image generator 515 may initially divide image data 510 into a plurality of sub-images where a grid may be placed on each sub-image and independently analyzed but those of ordinary skill in the related art will appreciate that sub-dividing the image may not always be necessary. For example, sub-dividing the image into sub-images provides for more accurate estimations of the position of each probe feature in the sub-image where, for instance, the degree of error of probe feature position estimation or registration in an image increases with distance from one or more positional reference points. In the present example, reference points may include control features, such as patterns of control probes, chrome features, or other fiducial features known in the related art that may include a checkerboard or other type of recognizable pattern. In some embodiments the control features may be positioned in the corners of the full array, and at the corner positions of each sub-array. By reducing the area of each sub-image and thus decreasing the distance from the control features that are positional references, the error associated with the positional estimation of probe feature location is reduced to an acceptable level. Additional examples of sub-dividing and grid placement on images is described in U.S. patent application Ser. No. 10/391,882, incorporated by reference above. Further, various types of positional reference features and their uses are also described in U.S. patent application Ser. No. 10/769,575, titled “System and Method for Calibration and Focusing a Scanner Instrument Using Elements Associated with a Biological Probe Array”, filed Jan. 29, 2004, which is also hereby by reference herein in its entirety for all purposes.

Sub-image generator 515 may produce a plurality of data files 517 for each sub-image for use by sub-image analyzer 570, employing one or more files from library files 274, image data file 510, one or ore experiment data files from experiment data 277. In some embodiments, sub-image generator 515 may employ a library file with sub-image details that for instance may include what may be referred to as a .SMD library file, where the sub-image details may comprise the number of sub-images to be generated and a measure of the degree of overlap between the sub-images. Similarly, sub-image generator 515 may employ another library file that includes details of probe array 140 that for instance may include what may be referred to as a .CIF library file, where the details of probe array 140 may comprise the numbers of rows and columns of probe features associated with the particular implementation of probe array 140, and an experiment file with experiment data that for instance may include what may be referred to as a .EXP data file, where the experiment data may comprise a measure of pixel size and the type of probe array 140. Also, sub-image generator 515 may place a grid on each of the sub-mages to provide positional registration of each of the probe features in the sub-image, where each probe feature may be bounded by the lines of the grid in what may be referred to as a cell. For example, sub-image generator 515 may sub-divide image data file 510 into 169 or more separate sub-images, place a grid on each sub-image, and produce a plurality of files 517 for each sub-image such as, for instance, sub-image file .dat 517A, sub-image .exp file 517B, and sub-image .cel file 517C.

In some embodiments, sub-image generator 515 may also produce an image file of the full image where a grid may be applied by generator 515. For example, sub-image generator 515 may produce full image .cel file 516 that may, in some embodiments, be employed for analysis of intensity values with respect to each of the probe features of probe array 140.

Image analyzer 570: Image analyzer 570 may receive each of files 517 and/or full image .cel file 516 for analysis. In the presently described embodiments, image analyzer 570 may employ one or more methods to assign a value of intensity for each cell in the image or sub-image being analyzed. For example, the analysis may include what may be referred to as a reconstruction analysis that uses a geometric model of probe array 140 to “reconstruct” the values for each probe feature cell with respect to the essential parameters of probe array 140 and the raw image values in files 516 or 517.

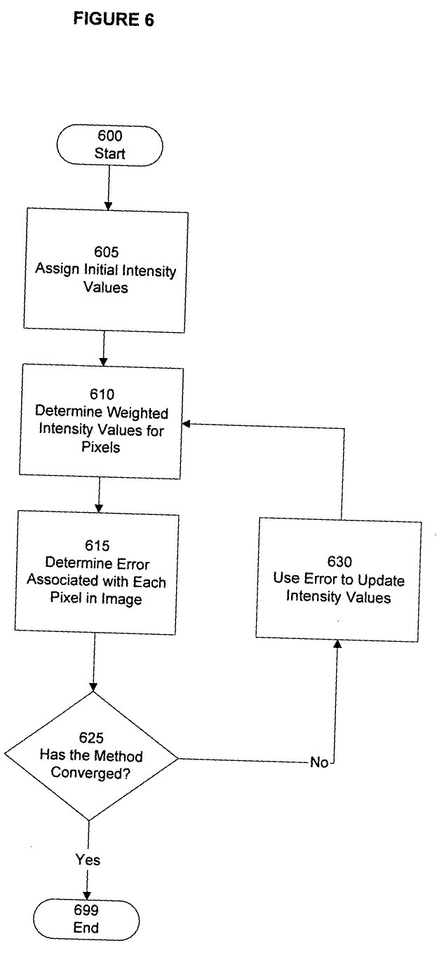

An example of a method that employs such a reconstruction model is provided in FIG. 6, where for example analyzer 570 may employ a geometric model of probe array 140 comprising the positional locations of probe features such as the locations of the associated cells that are defined by a grid where the position and orientation of each of the probe features on probe array 140 is known, for instance such information may be defined in one or more .CIF library files as described above. In the present example, a geometric model may be defined as an array of square cells that are uniform with respect to size and placement, where each cell is separated from its neighboring cells by boundary regions as they are referred to above. Also in the present example, the blur associated with the imaging process may also be modeled as a Gaussian function, or in the preferred implementation of analyzing images produced by a CCD type of optical architecture may comprise what may be referred to as an Airy function, where the functions may be referred to as a point spread function. Those of ordinary skill in the related art will appreciate that what may be referred to as the “Point Spread Function” (hereafter referred to as PSF) provides a measure of “blurring” from a single point object introduced into an image from an optical system such as for instance scanner 100. In the present example, the PSF may be described by a mathematical function that describes the optical distortion of the point source through the optical path of an instrument and may differ between instruments, as well as differing between image acquisition events in the same instrument. Also, an optical detection instrument such as scanner 100 may comprise different PSFs for different focal and/or spatial locations and further the PSF may not be a linear function.

In the example provided in FIG. 6, the following equation illustrates the relationship of the observed pixel intensity to a weight value and feature intensity:

$${g}_{i} = {\sum }_{j} {w}_{ij} {f}_{j} + {η}_{i} Image formation with noise η \qquad \underline{equation 1}$$

where:

gᵢ=observed pixel i

fⱼ=feature j

wᵢⱼ=weight value comprising the fraction of gᵢ's Point Spread Function overlapping fⱼ

Further, equation 1 may be expressed in Matrix form, solving for f; and error residuals are estimates of η:

$$\begin{matrix}{w}_{11} {f}_{1} + {w}_{12} {f}_{2} + ⋯ + {w}_{1 N} {f}_{N} = {g}_{1} \\ {w}_{21} {f}_{1} + {w}_{22} {f}_{2} + ⋯ + {w}_{2 N} {f}_{N} = {g}_{2} \\ ⋮ ⋮ \\ {W}_{M 1} {f}_{1} + {w}_{M 2} {f}_{2} + ⋯ + {W}_{MN} {f}_{N} = {g}_{M}\end{matrix} or Wf = g \qquad \underline{equation 2}$$

where:

M=number of pixels

N=number of features

For example, equation 2 is too large to solve directly, but may be solved by an iterative process. In the present example such an iterative process assigns an initial guess of the intensity values \hat{f}ⱼ for each cell representing a probe feature. The process then includes applying the imaging model (equation 2) to produce the reconstructed image that would result if the initial guess of the intensity values were true. This reconstructed image is compared to the image actually obtained by scanning probe array 140, and the difference is used to correct the estimates of the feature intensity values \hat{f}ⱼ.

In the presently described embodiments, analyzer 570 may initially assign a value of intensity for each cell in the reconstructed image defined by a grid, as illustrated in Step 605. In some embodiments, analyzer 570 may assign an arbitrary value, such as a value of zero, to each cell but it may be preferable to use a value that is more indicative of the actual measured intensity for that cell. For example, analyzer 570 may select an intensity value associated with the pixel positioned closest to the center of each cell in the raw image of files 516 or 517 as a representative measure of intensity for initial assignment for the corresponding cell in the reconstructed image.

Step 610 illustrates the step of determining weighted intensity values for each pixel associated with each cell representing each probe feature in the reconstructed image where the weights are dependent upon the degree to which the pixel overlaps the cell. For example, probe j will contribute a measure of intensity to pixel i according to the proportion of the dimension of the point spread function of pixel i that overlaps with probe j. In other words, the greater the degree of overlap of the point spread function, the greater the contribution of probe j will be to the intensity of pixel i. In the present example, weights may be modeled using the PSF, where for instance the weight may follow wᵢⱼ=the integral over probe feature/cell j of the PSF centered at pixel i.

Continuing with the present example, the weighted intensity value for each pixel in a given cell may be represented by:

$${t}_{i} = {\sum }_{j} {w}_{ij} {\hat{f}}_{j} \\ expected pixel i assuming value of feature \hat{f} \qquad \underline{equation 3}$$

where:

\hat{f} is the reconstructed intensity value for feature j that may include the initial intensity value or an updated/reconstructed value that will be described further below.

Subsequently, step 615 illustrates the step of determining an error value for every pixel in the image by subtracting the weighted intensity value for a given pixel from the measured intensity value of the corresponding pixel in the raw image of files 516 or 517. For example, the calculation of the error value may be given by:
\hat{η}ᵢ=gᵢ−tᵢ error term of pixel i equation 4

Also, analyzer 570 determines a value that is representative of the proportion of the error that is attributable to the cell representing the probe feature. In the present example, the determination may be given by:

$${c}_{ij} = \frac{{w}_{ij}}{{\sum }_{k} {w}_{ik}^{2}} {\hat{η}}_{i} \\ portion of pixel i error attributable to feature j \qquad \underline{equation 5}$$

As illustrated in decision element 625, if the equations have converged on a answer which may for instance include the error term \hat{ηᵢ} being below some threshold value that could be predefined or user selectable. Those of ordinary skill in the related art will appreciate that the term “convergence” generally refers to a sequence or series of steps that proceeds toward some limit, and that convergence is achieved when the steps have proceeded far enough to be within that limit, and thus any number of parameters defining such a limit may be employed. Continuing with the example of element 625, if analyzer 570 determines that convergence has been achieved, then the reconstructed intensity values for each cell is taken as representative of the actual measured intensity. Alternatively, if analyzer 570 determines that convergence has not been achieved, then the error values are used to update the feature intensity values as illustrated in step 630.

As stated above, Step 630 illustrates the step of using the error values to update the intensity values for each of the cells that represent the probe features. For example, analyzer may update the intensity values serially using:
\hat{f}ⱼ+=cᵢⱼ for eachj such that Wᵢⱼ>0 equation 6

In the present example, analyzer 570 may also update the intensity values in parallel using the following equation:

$${\hat{f}}_{j} += {\sum }_{i} \frac{{w}_{ij}}{{\sum }_{k} {w}_{ik}^{2}} ( {g}_{i} - {\sum }_{j} {w}_{ij} {\hat{f}}_{j}^{old} ) \qquad \underline{equation 7}$$

For example, updating serially causes faster convergence of the iterations but may include some error that is dominated by the last pixel to update the cell intensity. Therefore, it is preferable to employ the parallel update method for the last iteration of the method prior to convergence. In some embodiments, images of probe array 140 comprise high contrast and steep gradients of intensity values from cell to boundary area, where there may be an imperfect match between the model and probe array 140. In such cases it may be preferable to employ the parallel update for each iteration of the method.

Also in the present example, analyzer 570 may determine a value that represents the estimated error in the reconstructed/updated intensity value for each cell representing a probe feature. Those of ordinary skill in the related art will appreciate that such a value may be employed by analyzer 570 to determine convergence and the relative number of steps or iterations prior to convergence of the method. The estimation of error may be given by:

$${s}_{j}^{2} = \frac{{\sum }_{i} {w}_{ij} {\hat{η}}_{i}^{2}}{{\sum }_{i} {w}_{ij}} \qquad \underline{equation 8}$$

In the same or alternative implementations, the parallel update may work effectively for the first iteration, but for subsequent iterations a second parallel update model may be employed that performs more effectively and converges more quickly. For example, the second parallel update model includes re-weighted corrections according to the intensity of the pixels:

$${\hat{f}}_{j} += {\sum }_{i} \frac{{f}_{j} + noise\_floor}{{g}_{i} + noise\_floor} \frac{{w}_{ij}}{{\sum }_{k} {w}_{ik}} ( {g}_{i} - {\sum }_{j} {w}_{ij} {\hat{f}}_{j}^{old} ) \qquad \underline{equation 9}$$

As described above with respect to equations 6-9, analyzer 570 determines a correction term from the difference between the intensity values from the acquired image, and the reconstructed intensity values for the cells representing the probe features of the reconstructed image, where analyzer 570 adds the correction term back into the intensity value during the iterative update.

An alternative multiplicative approach could be employed in some embodiments that comprises analyzer 570 computing a ratio value of the raw intensity values from the acquired image to the reconstructed intensity values for the cells representing the probe features of the reconstructed image that leads to a set of corrective factors. Analyzer 570 then multiplies the corrective back into the reconstructed feature values during the iterative update. This approach of multiplicative updating may for example be given by:

$${u}_{i} = \frac{{g}_{i}}{{\sum }_{j} {w}_{ij} {f}_{j}} \qquad \underline{equation 10}$$

where uᵢ represents the ratio value

$${v}_{j} = {\sum }_{i} {w}_{ij} {u}_{i} \qquad \underline{equation 11}$$

where vⱼ represents the corrective factor to be multiplied back into \hat{f}

In the presently described example, the initially assigned intensity value described with respect to step 605 for each cell is preferably greater than zero. For instance, if an initial intensity value of zero is employed, the result of the iterative process will not be able to update the intensity value to a more accurate value.

In addition, it may be desirable in some implementations for analyzer 570 to perform one or more methods to improve the accuracy of registered reconstructed features and pixels, as well as to correct for error created by characteristics of scanner 100 such as, for instance blurring or waviness in the image produced by non-uniform motion of the excitation spot relative to probe array 140. For example, image analyzer 570 may measure how much the image has been shifted to the left or right; or vertically up or down using a line of pixel intensity information in the respective axis.

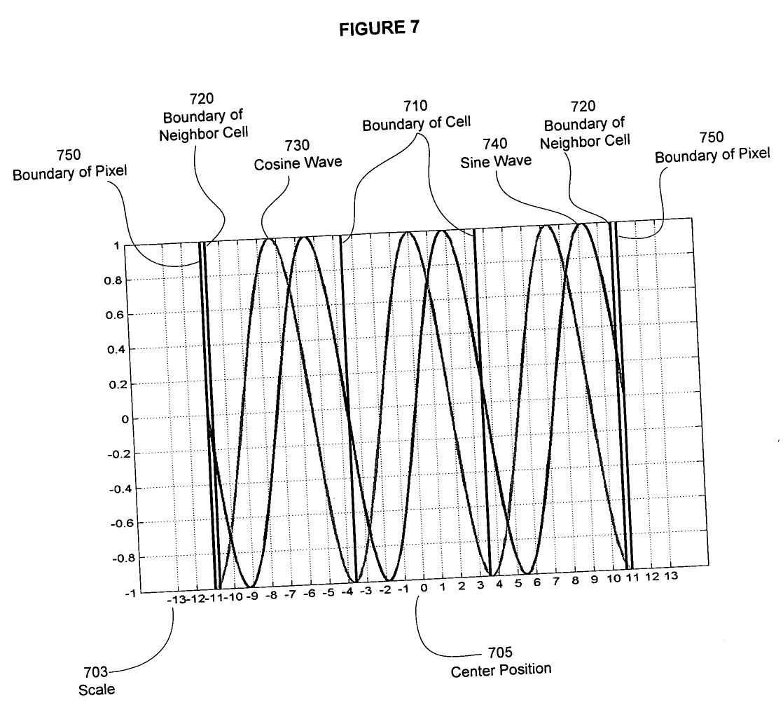

For example, one possible approach to measuring these shifts is illustrated in FIG. 7. This is drawn for the case that probe features are measured on 5 μm centers, imaged with pixels comprising a 0.7 μm pitch. Then one cell spans 5 μm/0.7 μm=7.14 pixels. For reasons of continuity, noise rejection, and reduction of edge effect, the shift is computed over a window spanning three cell widths, although more or fewer cell widths could certainly be employed. Three times the cell width is 3×7.14=21.43 pixels, which we raise to 22 pixels so as to work with whole pixels. Since 22 is an even number, the window center will fall on a pixel boundary, with 11 pixels to the left and 11 to the right. (If the window had been rounded up to an odd number, then the window center would have fallen in the middle of the center pixel.) Therefore, FIG. 7 is drawn with pixel scale 703 showing the distance from the center of the window, in pixels; 705 showing the center of the window; 710 showing the boundaries of the cell under scrutiny; 720 showing the outer boundaries of the neighboring cells; and 750 showing the extremities of the window of pixels being called into play. Over the pixel window is imposed an In-phase cosine wave 730, and also a sine wave 740, each with period equal to the cell width. We take the inner product of the cosine wave with the pixels to produce an In-phase (I) signal. The inner product of the sine wave with the pixels similarly produces a Quadrature (Q) signal.

In the present example, the image is expected to be relatively bright at cell centers, and dark at the boundaries between cells. Therefore, if the cell is indeed positioned at position 0 on the scale (705), the I signal will be high and the Q signal near zero. If the cell is shifted off to one side, I will fall, while Q will rise if the shift is to the right, or fall if the shift is to the left. Therefore, we can measure the amount of shift of the image against the window by calculating the arctangent of Q/I. To calculate the shift in pixels, we use the formula:

$$shift = \frac{1}{2 π} {tan}^{- 1} ( \frac{Q}{I} ) \times \frac{cell pitch}{pixel pitch} \qquad \underline{equation 10}$$

This shift is of course relative to the center of the window. The expected position of the cell center is most likely not at the center of the window, but some fraction of a pixel offset from it. Therefore, the shift calculated over the window must be corrected to the expected center.

Continuing with the present example, to reduce sensitivity to noise, the pixel window may be more than one pixel wide in an orthogonal direction. This also makes the shift calculation more robust against an unexpected shift in the orthogonal direction. In this example, when measuring the horizontal shift, the window may be 22 pixels wide by 3 pixels high (or 5 or some other number). The pixels are summed vertically before being applied to the sine and cosine waves. The sums may be weighted sums, so that the expected vertical position is preferred.

For further rejection of noise and contaminants (e.g., dust specks), the shifts may be smoothed together in a region. This can be done using the shift values, but it is preferable to smooth the I and Q values before taking the arctangent. If, say, one scan line may be shifted up or down against its neighbors, the I and Q values measured vertically may be smoothed horizontally; and if the columns may be shifted laterally, then the I and Q waves measured horizontally may be smoothed vertically.

Image collator 580: In some embodiments, image collator may then receive each re-constructed sub-image and collates them into a single reconstructed image of probe array 140, such as results data file .cel 590. In some embodiments, collator 580 may also delete or remove sub-image files 517 after the collation step. Also, in some embodiments .cel file 590 may be presented to a user in one or more of GUI's 246, be stored in one or more data structures or files, or may be employed directly in further analysis methods.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments.

Also, the functions of several elements may, in alternative embodiments, be carried out by fewer, or a single, element. Similarly, in some embodiments, any functional element may perform fewer, or different, operations than those described with respect to the illustrated embodiment. Also, functional elements shown as distinct for purposes of illustration may be incorporated within other functional elements in a particular implementation. Also, the sequencing of functions or portions of functions generally may be altered. Certain functional elements, files, data structures, and so on may be described in the illustrated embodiments as located in system memory of a particular computer. In other embodiments, however, they may be located on, or distributed across, computer systems or other platforms that are co-located and/or remote from each other. For example, any one or more of data files or data structures described as co-located on and “local” to a server or other computer may be located in a computer system or systems remote from the server. In addition, it will be understood by those skilled in the relevant art that control and data flows between and among functional elements and various data structures may vary in many ways from the control and data flows described above or in documents incorporated by reference herein. More particularly, intermediary functional elements may direct control or data flows, and the functions of various elements may be combined, divided, or otherwise rearranged to allow parallel processing or for other reasons. Also, intermediate data structures or files may be used and various described data structures or files may be combined or otherwise arranged. Numerous other embodiments, and modifications thereof, are contemplated as falling within the scope of the present invention as defined by appended claims and equivalents thereto.

What is claimed is:

1. A method of reconstructing an image of a biological probe array, comprising:
(a) receiving a raw image of a biological probe array comprising a plurality of cells that is each representative of a probe feature on the probe array, wherein each cell comprises a plurality of pixels each comprising a raw intensity value;
(b) assigning an intensity value to each of a plurality of reconstructed cells of a reconstructed image, wherein each reconstructed cell comprises a plurality of reconstructed pixels;
(c) determining a weighted intensity value for each reconstructed pixel in each reconstructed cell using the intensity value of the reconstructed cell and a weight value;
(d) determining an error value for each reconstructed pixel using the weighted intensity value and the raw intensity value of a corresponding pixel in the raw image;
(e) updating the intensity value of each of the reconstructed cells using the error value; and
(f) repeating steps (c)-(e) until convergence, wherein the intensity value for the reconstructed cells of the converged reconstructed image is representative of light emitted from the corresponding probe features.

2. The method of claim 1, wherein:
the raw image comprises blurring error.

3. The method of claim 2, wherein:
the blurring error is associated with a point spread function of an optical instrument.

4. The method of claim 2, wherein:
the blurring error is modeled using a Gaussian point spread function.

5. The method of claim 2, wherein:
the blurring error is modeled using an Airy point spread function.

6. The method of claim 1, wherein:
the raw intensity value for each pixel comprises a measure of detected light from the biological probe array.

7. The method of claim 1, wherein:
the cells of the raw image are defined by a grid.

8. The method of claim 7, wherein:
the grid comprises vertical and horizontal lines that bound the cells.

9. The method of claim 7, wherein:
the grid provides positional registration of the cells that represent probe features.

10. The method of claim 1, wherein:
the assigned intensity value for each reconstructed cell comprises the raw intensity value of a pixel positioned closest to the center of each corresponding cell in the raw image.

11. The method of claim 1, wherein:
the weight value is dependent upon the degree to which the reconstructed pixel overlaps the reconstructed cell.

12. The method of claim 11, wherein:
the degree to which the reconstructed pixel overlaps the reconstructed cell is determined using a point spread function of an optical system.

13. The method of claim 1, wherein:
the error value comprises the weighted intensity value for the reconstructed pixel subtracted from the raw intensity value of the corresponding pixel in the raw image.

14. The method of claim 13, further comprising:
determining a measure of error attributable to the reconstructed cell using the error value, wherein the measure of error is employed in the step of updating.

15. The method of claim 13, wherein:
the step of updating comprises a parallel update.

16. The method of claim 1, wherein:
the error value comprises a ratio value of the raw intensity values for a cell to the reconstructed intensity values of the corresponding reconstructed cell.

17. The method of claim 16, further comprising:
determining a corrective factor using the ratio value.

18. The method of claim 16, wherein:
the step of updating comprises a multiplicative update.

19. A method of reconstructing a cell in using a raw image of a biological probe array, comprising:
(a) assigning an intensity value to a reconstructed cell of a reconstructed image, wherein each reconstructed cell comprises a plurality of reconstructed pixels;
(b) determining a weighted intensity value for each reconstructed pixel in the reconstructed cell using the intensity value of the reconstructed cell and a weight value;
(c) determining an error value for each reconstructed pixel using the weighted intensity value and a raw intensity value corresponding to a pixel in the raw image;
(d) updating the intensity value of the reconstructed cell using the error value; and
(e) repeating steps (b)-(d) until convergence, wherein the intensity value for the reconstructed cell is representative of light emitted from a corresponding probe feature on the biological probe array.

20. The method of claim 19, wherein:
the raw image comprises blurring error.

21. The method of claim 20, wherein: the blurring error is associated with a point spread function of an optical instrument.

22. The method of claim 20, wherein:
the blurring error is modeled using a Gaussian point spread function.

23. The method of claim 20, wherein:
the blurring error is modeled using an Airy point spread function.

24. The method of claim 19, wherein:
the raw intensity value for each pixel comprises a measure of detected light from the biological probe array.

25. The method of claim 19, wherein:
the assigned intensity value for the reconstructed cell comprises the raw intensity value of a pixel positioned closest to the center of a corresponding cell in the raw image.

26. The method of claim 19, wherein:
the weight value is dependent upon the degree to which the reconstructed pixel overlaps the reconstructed cell.

27. The method of claim 26, wherein:
the degree to which the reconstructed pixel overlaps the reconstructed cell is determined using a point spread function of an optical system.

28. The method of claim 19, wherein:
the error value comprises the weighted intensity value for the reconstructed pixel subtracted from the raw intensity value of a corresponding pixel in the raw image.

29. The method of claim 28, further comprising:
determining a measure of error attributable to the reconstructed cell using the error value, wherein the measure of error is employed in the step of updating.

30. The method of claim 28, wherein:
the step of updating comprises a parallel update.

31. The method of claim 19, wherein:
the error value comprises a ratio value of the raw intensity values for a cell to the reconstructed intensity values of the corresponding reconstructed cell.

32. The method of claim 31, further comprising:
determining a corrective factor using the ratio value.

33. The method of claim 31, wherein:
the step of updating comprises a multiplicative update.
